(19) United States
(12) Patent Application Publication
(10) Pub. No.: US 2007/0037885 A1
(43) Pub. Date: Feb. 15, 2007

(54) Biomass containing animal feed

(76) Inventor: Sandra Edwards, Newcastle Upon Tyne (GB)

Correspondence Address: KLARQUIST SPARKMAN, LLP, 121 SW SALMON STREET, SUITE 1600, PORTLAND, OR, 97204, US

(21) Appl. No.: 10/554,493

(22) PCT Filed: Apr. 22, 2004
(86) PCT No.: PCT/GB04/01724

§ 371 Date: Sep. 6, 2006

(30) Foreign Application Priority Data
Apr. 25, 2003 (GB) 0309453.9
May 8, 2003 (GB) 0310521.0

(51) Int. Cl. A61K 31/202; A23K 1/165; A61K 36/02

(52) U.S. Cl. 514/560; 424/442; 424/195.17

(57) ABSTRACT

We describe a method to increase the viability, weight, development and behaviour of animals comprising administering to a pregnant animal a long chain fatty acid such as docosahexaenoic acid.

The invention relates to a method to improve the viability, weight, development and behaviour of animals; and including animal feed comprising polyunsaturated fatty acids, for example docosahexaenoic acid (DHA) for administration to pregnant mammals.

DHA, an example of a n-3 fatty acid, can be obtained directly from the diet or derived from metabolism of dietary linoleic and α-linolenic acid. However, in the latter case, the metabolic conversion rate is poor. To obtain sufficient amounts of this fatty acid animals have to eat foods rich in DHA. Currently the principle dietary source of DHA is fish or fish oil. However, this has many inherent problems; fish accumulate pollutants, for example PCBs, the extracted oil has an unpleasant odour, there is a difficulty in controlling the proportion of specific desirable fatty acids from this source. Fish are a declining resource and the market demand for DHA is not being met. Also, vegetarians do not have an obvious alternative food source to fish and therefore either do without DHA or have to take pure supplements.

During evolution humans have consumed a diet containing an approximately equal ratio of n-3 and n-6 essential fatty acids (1-2:1), but the last 100-150 years has seen a growing trend in Western diets towards the consumption of more n-6 fatty acids, resulting in an alteration of the ratio to 30:1 (Simopoulos, Am. J. Clin. Nutr. 1999, 70 (3); 560S-569S). Whilst an increased intake of n-6 fatty acids is characterised by cardiovascular problems such as increased blood viscosity, vasospasm and vasoconstriction, the n-3 fatty acids are associated with health promoting properties. For example, n-3 fatty acids have been described as anti-inflammatory, antithrombotic, antiarrhytrnic, hypolipidemic and vasodilatory (Simopoulos, 1999). As such the role of DHA in the prevention and/or treatment of diseases such as coronary heart disease, hypertension, type II diabetes, ocular diseases, arthritis, cystic fibrosis and schizophrenia and has been the focus of a great deal of medical research.

Essential fatty acids are structural components of all tissues and are indispensable for cell membrane synthesis. The brain, retina and other neural tissues have been found to be particularly rich in DHA where it is involved in neural development and maturation of sensory systems (Uauy et al. Proc Nutr Soc 2000 Feb;59(1):3-15). DHA forms 25% of the fatty acid complement of the glycosphingolipids of the brain and is an important component of the rods of the retina, and therefore a deficiency in DHA during infant development has been associated with a reduction in cognitive function and visual acuity. Furthermore, deficiencies in DHA have been associated with foetal alcohol syndrome, attention deficit hyperactivity disorder, cystic fibrosis, phenylketonuria and adrenoleukodystrophy.

To meet the increased demand for n-3 fatty acids, such as DHA, a number of approaches have been attempted. The cultivation of marine micro-organisms such as the Crypthecodinium cohnii and Schizochytrium spp, which are rich sources of DHA, has met with some success but the cultivation of algae is technically demanding and costly (Ashford et al., Lipids 2000 Dec;35(12):1377-86). U.S. Pat. No. 5,711,983, that is incorporated by reference in its entirety, discloses a method to optimise the production of DHA in edible algal species, in particular Crypthecodinium cohnii, by the manipulation of fermentation growth conditions. By doing so it is possible to achieve high yields of DHA in culture. Further examples of the use of algal sources for DHA are described in EP0515460, U.S. Pat. No. 5,407,957, EP0512997, U.S. Pat. No. 6,451,567 and WO02/092540.

It is known that long chain fatty acids such as DHA are involved in early mammalian development. Currently, the sources of DHA are provided either as fish oil, with its attendant disadvantages as detailed above, or directly from edible algae. The long chain fatty acid delivered as a nutritional supplement as described herein may be in a form such as, but not limited to, a free fatty acid, or an ester thereof such as a triglyceride, diglyceride, monoglyceride, phospholipids, glycolipid, sulpholipid or sphingolipid.

As mentioned above the provision of DHA as a nutritional supplement to the mammalian diet has had some success but can be improved upon. For example, WO97/49297 describes the addition of both DHA and an inhibitor of microbial degradation to obtain milk containing elevated levels of DHA. EP1106076 describes the a method to reduce the exudation of meat juice from meat comprising the feeding of livestock a plant extract from Stevia spp combined with fish meal as a source of DHA. The meat of pigs that have been fed the combined fish meal/plant extract have a lower fat content and contain DHA.

We disclose a method for the administration of long chain fatty acids to a diet that enhances behavioural and physical development of mammals. We have surprisingly found that to maximise the effect of long chain fatty acids on mammalian development the fatty acid has to be delivered to the mammal during a particular period of growth. We have investigated the pre and post-natal effects of supplementation of sow diet with essential long chain fatty acids. We show that supplementation of the sow or sheep diet with, for example DHA, results in piglets or lambs that show increased post-weaning weight, which has been associated in previous work with improved health and behavioural patterns. In particular, the supplementation of sow diet with DHA during late gestation improves piglet viability; feed intake/growth rate after weaning; and final piglet weight at around 8 weeks of age. The addition of DHA during lactation improves piglet weaning weight.

According to an aspect of the invention there is provided a method to improve the development of an animal species comprising;

i) administering to said animal species at least one long fatty acid wherein said fatty acid is provided during late gestation and/or during lactation; and

ii) allowing an infant of said species to suckle on said animal administered said long chain fatty acid.

In a preferred method of the invention said infant is the progeny of said animal.

In an alternative preferred method of the invention said infant is not the progeny of said animal.

It will be apparent that gestation periods for different mammals will vary and will be apparent to the skilled person, (for example pig 115 days, sheep 149 days, horse 340 days).

In a preferred method of the invention said animal is a mammal, preferably a livestock species. Preferably said mammal is selected from the group consisting of: cow; sheep; goat; horse; mink; or a pig Preferably, said mammal is a pig or sheep. More preferably still said mammal is a pregnant pig or sheep.

In a further preferred method of the invention said mammal is a companion mammal. Preferably said companion mammal is selected from the group consisting of: dog; cat; hamster; mouse; rabbit; pot bellied pigs; rat; gerbil; or guinea pig.

In a further preferred method of the invention said long chain fatty acid is a free fatty acid, or an ester thereof.

Preferably said free fatty acid is selected from the group consisting of: a triglyceride, diglyceride, monoglyceride, phospholipids, glycolipid, sulpholipid or sphingolipid.

In a further preferred method of the invention said long chain fatty acid is docosahexanoic acid.

In a preferred method of the invention docosahexanoic acid is provided as a supplement to animal feed. Preferably said docosahexanoic acid is provided in said animal feed as an edible algae, for example Crypthecodiniurn cohnii, as disclosed in U.S. Pat. No. 5,711,983 or Schizochytrium spp as described in EP0512997.

It will be apparent to the skilled person that a variety of sources of DHA can be provided, for example fish meal, or as an algal form, for example an algal DHA supplement (supplier Advanced BioNutrition Corporation).

In a further preferred method of the invention said docosahexaenoic acid is provided in the late gestation period, preferably about the last 4 weeks of gestation for the pig and preferably about the 12^{th} to the 18^{th} week of gestation for the sheep.

In an alternative preferred method of the invention said docosahexaenoic acid is provided during the lactation period. Preferably said docosahexaenoic acid is provided during the period up until weaning.

In a preferred method of the invention said mammal is a pregnant pig or sheep. Preferably docosahexaenoic acid is provided to said pig or sheep at least once daily during late gestation, preferably once daily.

In a further preferred method of the invention docosahexaenoic acid is provided at least once daily during the lactation period, preferably twice daily

According to a further aspect of the invention there is provided the use of at least one long chain fatty acid for the manufacture of an animal feed for use during late gestation and/or during lactation of a mammal.

In a preferred use of the invention said long chain fatty acid is docosahexanoic acid.

In a further preferred use of the invention said docosahexaenoic acid is provided as an edible algae. Preferably said edible algae is selected from the group consisting of: Crypthecodinium cohnii; Phaedactylum spp; Isochrysis spp; or a chytrid e.g. Schizochytrium spp; Thaustochytrium spp; or Ulkenia spp.

According to a further aspect of the invention there is provided a method to improve the development of a poultry species comprising; administering to said species at least one long chain fatty acid. Preferably, said fatty acid is provided during late gestation.

An embodiment of the invention will now be described by example only and with reference to the following tables;

Table 1 illustrates litter performance during lactation of sows fed DHA supplement in the last 4 weeks of gestation or during a 4 week lactation;

Table 2 illustrates post-weaning performance of litters from sows fed DHA supplement in the last 4 weeks or during a 4 week lactation; and

Table 3 illustrates a Crypthecodinium cohnii biomass composition used in the feeding experiments detailed below;

Table 4 illustrates the effect of feeding long chain fatty acids to pregnant sheep and the effect on lamb viability;

Table 5 illustrates the effect of feeding long chain fatty acids to ewes prior to giving birth;

Table 6 illustrates the effect of feeding long chain fatty acids to ewes on lamb performance; and

Table 7 illustrates the effect of feeding long chain fatty acids to ewes on lamb behaviour.

Materials and Methods

Experimental Design and Treatments (Pigs)

The experiment was designed as a 2×2 factorial with DHA supplementation of the sow diet (3 g DHA/kg feed) during the last 4 weeks of pregnancy and/or 4 weeks of lactation. There were 8 replicate sows/litters for each treatment combination.

Animals

32 crossbred sows (Landrace×Large White×Duroc, JSR Healthbred Ltd) were selected in four time blocks of 8 aninmals with contemporary farrowing dates. Sows had been inseminated with Large White semen (JSR Healthbred Ltd) and were selected 4 weeks prior to expected farrowing date. Allocation was such that treatments were matched for sow parity.

Procedures

Pregnant sows were housed in groups of 4-5 animals in straw-bedded, kennelled accommodation with individual feeding stalls. They were individually fed, once daily, with a standard feed allowance of 3 kg/day. A standard home-mix gestation diet (no fishmeal, see Table 1) was used, and was top dressed with either a DHA supplement, see Table 3 (26.7 g/kg supplement of which approximately 14.7 g is algal biomass containing approximately 3 g pure DHA/kg), or a control supplement containing maize oil (6 g/kg), profine soya isolate (13.3 g/kg) soyabean meal (5.3 g/kg) and vitamin/mineral supplement (1.3 g/kg) to equalise intake of DE, protein and micronutrients.

At -5 days before their expected farrowing date, sows were transferred to farrowing crates in part-slatted pens. They continued to receive the same pregnancy ration until farrowing, at which point they were changed to a lactation ration with the appropriate supplement added at the same rate per kg feed as in gestation. The ration was a standard home-mix lactation ration (no fishmeal, see Table 1). This was fed twice daily on a set scale increasing with day after farrowing. Sows received 3 kg on the day of farrowing, with allowance increasing by 0.5 kg/day provided that the previous day's feed has been completely consumed. If a small amount of refusal was left, the allowance was maintained, and if significant refusal was left (>0.5 kg) the allowance was reduced by 1 kg. Any feed refusals were collected, weighed and a sample taken for dry matter determination. Day 1 was designed as the first day on which piglets were present, and feed levels were changed prior to afternoon feeding. Water was freely available from a drinker in the sow trough. Piglets were individually identified at birth using ear tags. Piglet management routines (teeth clipping, iron injection, tail docking) were carried out within the first 36 hours of life according to standard unit commercial practice. Cross fostering was minimised, but was permitted where necessary to safeguard the welfare of the piglets. Wherever possible, cross-fostering was then carried out within 24 h of birth and within treatment or with non-experimental sows. Piglets were offered a commercial creep feed (containing no fishmeal; Primary Select, Primary Diets Ltd) from 10 days of age.

Piglets were weaned on a Thursday at -4 weeks of age and moved to flat deck accommodation where they were penned in treatment groups of 5 or 6 pigs (depending on the pen size in the flat deck room for that week's allocation). Each litter provided one pen of pigs for behaviour study, selected by ranking the pigs by weight and taking the male and female closest to the upper and lower inter-quartile weight (4 pigs) plus the median pig (or both male and female pigs, depending on pen size). The remaining pigs were penned by treatment (selecting pigs closest to median weight where excess animals were available), to give a further mixed-litter pen for each of the four treatment combinations. These pens were performance recorded but not subject to behaviour study. This gave a total of 3 pens per treatment within each time block. Pigs were offered a standard commercial starter diet to appetite for the first 7 days after weaning (Primary Select, Primary Diets Ltd), followed by a standard weaner diet for the next 3 weeks Primary Excell, Primary Diets Ltd), neither diet contained fishmeal or any DHA source. All pigs were individually weighed at weaning and 7, 14, 21 and 26 days after weaning, at which point pigs left the flat decks and the experiment terminated. Feed added to the hoppers was recorded and refusals weighed back on days 7, 14, 21 and 26.

1. Experimental Design and Treatments (Sheep)

4 treatment groups:

1: Control: No biomass inclusion in the diet.

2: 3-weeks inclusion: Biomiass inclusion (64 g/head/day) in the diet from 9 weeks prior to expected lambing date up until 6 weeks before lambing

3: 6-weeks inclusion: Biomass inclusion (64 g/head/day) in the diet from 9 weeks prior to expected lambing date up until 3 weeks before lambing.

4: 9-weeks inclusion: Biomass inclusion (64 g/head/day) in the diet from 9 weeks prior to expected lambing date up to lambing

The periods of biomass inclusion were selected with reference to the timing of neural development of the foetus.

Diets

Biomass was provided and ewe feed was mixed on farm. Control diets were supplemented with a source of vegetable oil and molasses to equate the fat, protein, carbohydrate and water contents of the two diets. Concentrate feeding level was on a per head per day scale relating to stage of gestation.

Procedures

1. 48 North of England Mule ewes, with known conception date and scanned as carrying twins, were allocated between treatments on the basis of conception date and sire breed.

2. They were housed in an open fronted strawed shed in 4 treatment groups of 12 ewes. At lambing ewes were moved to individual pens, where they stayed with their lambs until after the 24 h bonding period.

3. Grass silage was offered ad libitum. Silage feed intake measurements were taken daily during the experimental period by weighing the level of silage offered, and the level remaining in the trough prior to the next feed. Samples of silage, ewe-feed and biomass were also taken weekly throughout the trial.

4. Ewe blood samples were obtained prior to experimental diet inclusion, after experimental diet inclusion and on a 3 weekly basis thereafter.

5. As predicted lambing date approached, ewes were monitored on a 24 hr basis. Lambing was attended and assistance given if required, according to good farming practice.

6. As soon as lambs were born, they were identified by distinguishing features and a 5 cm sample of umbilical cord was taken and frozen. The time of lambing was recorded and a colostrum sample was collected from the ewe.

7. Lambs were monitored during the maternal bonding period and the time recorded when each lamb first stood (on all four feet for more than 10 seconds).

8. Each lamb was then removed from the pen, weighed and blood sampled. Lambs were also ear-tagged for identification at this point. They were then returned to their mother and allowed to suckle naturally.

9. Ewes which had lambed were blood sampled immediately prior to the next concentrate feeding time.

10. At 24 hours after birth, lambs were weighed and blood sampled again.

11. Lamb-weights will be obtained at intervals until weaning.

12. Colostrum and plasma samples at birth and 24 h will be analysed for fatty acid profiles. Colostrum and 24 h lamb serum samples will be analysed for immunoglobulin levels.

2. Experimental Designs and Procedures (Sheep)

The project used 200 ewes from a ram synchronisation study, since these had known tapping dates to allow calculation of gestation length. Only ewes scanned as carrying multiple fetuses were selected.

1. The experimental ewes were split into two flocks balanced for previous synchronisation treatment (ram exposure or not), lambing date and litter size.

2. They were housed in 4 groups of 50 ewes. Two groups were allocated to the experimental feed supplementation and the others remained as unsupplemented controls.

3. The sheep were fed according to normal farm practice, with the normal farm concentrate being fed at a specified, rising allowance per head in the period up to lambing.

4. The experimental supplement was given from four weeks before the predicted first date of lambing (29 Mar. 2003).

5. The experimental supplement (algal biomass, Advanced BioNutrition Europe Ltd Ltd) was fed at 40 g/head/day and was top-dressed onto the concentrate feed to ensure maximum probability of equal distribution.

6: Ewes lambed under 24-hour supervision, although intervention during lambing only took place if required according to normal commercial practice for ewe and lamb welfare. At lambing, the ewes were moved immediately to a single pen and basic performance data (listed below) were recorded for all ewes in the control and experimental groups.

7. In addition, neonatal behavioural data were recorded for as many litters as possible given the pattern of lambing. All lambs within a selected litter were recorded.

Statistical Analyses

Data were analysed by two-way analysis of variance using gestation treatment (+/−DHA) and lactation treatment (+/−DHA) as main factors, and their interaction. For lactation analyses, the litter was used as the statistical unit. For post-weaning analyses; the pen of piglets was used as the statistical unit. Time replicate was used as a blocking factor. Covariates of litter size, weaning age and weaning weight were tested and used as appropriate.

### EXAMPLE 1

Lactation Performance

Table 1 summarises the litter performance during lactation. Total litter size at birth differed between treatments, although this was a random effect and not a treatment consequence since sows were allocated only in late gestation. The number of piglets born dead was attributable to the differences in total litter size, and no significant treatment difference existed when litter size was used as a covariate in the model. The total litter weight at birth also differed between treatments, but this was a reflection of differences in litter size. Individual piglet birthweight did not differ between treatments when litter size was included as a covariate in the model.

Fostering was carried out when necessary to reduce litters to correspond with the number of functional teats available. This gave smaller and non-significant differences in litter size at the start of lactation. However, since the absolute litter size differed between treatments by up to one piglet, initial litter size was used as a covariate in all subsequent analyses. The number of piglets which died during lactation was relatively high, averaging 12.4%. This, and the high initial litter size, were a reflection of the relatively high age structure of the herd at this time, as a result of suspended replacement of breeding stock during the Foot & Mouth disease outbreak. There was a tendency (P=0.15) for mortality to be lower in litters which had received the DHA supplement during gestation, and this was still the case (P=0.12) when litter size was fitted as a covariate in the model. This is in accord with earlier results obtained from DHA supplementation with fish oil during gestation (Rooke et al., 2001), but a greater number of litters would be required for definitive evaluation of effects on this parameter, which has a high coefficient of variation.

The number of pigs weaned did not differ significantly between treatments and neither did the total litter weaning weight, although there was a tendency (P=0.11) for this to be greater in litters that had received DHA supplement during lactation. Age at weaning showed no significant difference between treatments although a mean difference was apparent because of the need to batch wean all litters into the post weaning phase on the same day. Both litter size and weaning age were therefore fitted as covariates (and exerted significant effect P<0.01) in the model to compare the individual weaning weight of piglets. This showed a significant positive effect (P<0.05) of lactation DHA on weaning weight.

### EXAMPLE 2

Post Weaning Performance

Piglets from sows which had received DHA supplement in gestation were heavier throughout the post-weaning period than controls (P<0.05), as were piglets whose mothers received DHA in lactation (P<0.05). After correction for differences in age and weight at weaning, the live weight gain in week 1 and over the whole 26 day trial period was significantly greater for piglets from sows which had received DHA supplement in gestation. There was no significant effect of lactation supplement. The effect on live weight gain was partially attributable to improved feed intake. Piglets from sows which had received DHA supplement in gestation has greater feed intake in week 1 (P<0.05) and a tendency for greater intake over the whole period (P=0.12). The week 1 effect was reduced, but still apparent (P=0.07), after correction for age and weight at weaning. However, piglets from sows which had received DHA supplement in gestation also showed a tendency for better feed efficiency, both in the first week and overall (P=0.08). Again, the week 1 effect was reduced, but still apparent (P=0.10), after correction for age and weight at weaning.

### EXAMPLE 3

Behaviour After Weaning

Analyses of video tapes (a total of 8448 recorded pig hours) for behavioural data is still in progress and no results are available as yet.

DHA supplementation of sows in late gestation is likely to improve piglet viability (based on the trends in this study and previous trial results). However, a larger scale experiment would be required for confimnation. DHA supplementation of sows in lactation improves piglet weaning weight. DHA supplementation of sows in late gestation improves piglet feed intake and growth rate after weaning. DHA supplementation of sows in late gestation and in lactation both improve final piglet weight at 8 weeks of age.

### EXAMPLE 4

The Effect of a Source of Omega-3s in Breeding Ewe Diets on Measures of Lamb Viability.

Lamb mortality generates a significant financial loss to the agricultural sector. The possibility of increasing lamb viability by nutritional means during gestation has been widely explored in numerous species. Recently investigations have turned to the potential of using long chain omega-3 essential fatty acids (EFAs) in maternal diets during late pregnancy. The thinking behind such work lies with the knowledge that these are the major fatty acids in brain and nervous tissue, and therefore have specific roles in neural development and cognitive function. Many of these investigations have used fish oil as the primary source of omega-3 EFAs, with little work being done in ruminant species. Lines 15 to here would be better integrated into the introduction. We examined the effects of feeding biomass to pregnant ewes on measures of lamb viability and investigated the influence of period of inclusion of biomass in the ewe diet, prior to lambing. The results are illustrated in Table 4 below.

TABLE 4

| | | 3 | 6 | 9 | | |
|---|---|---|---|---|---|---|
| | Cntl | weeks | weeks | weeks | SEM | Sig |
| Gestation | 145.2 | 147.1 | 147.5 | 148.0 | 0.77 | P = 0.08 |
| Length (d) | | | | | | |
| Birth wt (kg)≅ | 5.34 | 5.32 | 5.12 | 5.10 | 0.16 | NS |
| Time to | 31.02 | 27.87 | 21.63 | 22.61 | 2.8 | P = 0.037 |
| stand (min)= | | | | | | |
| Wt gain in | 0.28 | 0.17 | 0.03 | 0.30 | 0.10 | NS |
| first 24 h (kg) | | | | | | |

≅with gestation length as a covariate (P < 0.001)
=with adjustment for level of assistance during birth

### EXAMPLE 5

Data were successfully obtained from 172 lambings (89 treatment ewes and 83 controls). Treatment and control flocks did not differ in ewe condition score or litter size (Table 5). Ewes receiving the DHA supplement had significantly longer gestation length (P<0.01). There was no effect of treatment on mortality during lambing, or on mortality of lambs born alive, which were both low (<3%).

TABLE 5
Ewe data at lambing

| | Control | +DHA | sem | Sig |
|---|---|---|---|---|
| N | 83 | 89 | | |
| Ewe condition score | 1.40 | 1.34 | 0.1 | ns |
| Gestation length (d) | 146.2 | 147.8 | 0.64 | 0.003 |
| Litter size | 2.17 | 2.19 | 0.60 | ns |
| Ewes with lambs born dead | 4/83 | 7/89 | | ns |
| Ewes with later lamb deaths | 5/83 | 5/84 | | ns |

Lamb weight at 6 h and 24 h after birth did not differ between treatments, nor did liveweight gain over this period (Table 6). This would indicate no significant difference in colostrum/milk availability. Treatment lambs were lighter at weaning (P<0.05), but were also weaned on average one day younger than controls. The daily liveweight gain between birth and weaning did not differ between treatments.

TABLE 6
Lamb performance data

| | Control | +DHA | Sem | Sig |
|---|---|---|---|---|
| N | 176 | 189 | | |
| Lamb weight at 6 h (kg) | 5.07 | 5.11 | 0.11 | ns |
| Lamb weight at 24 h (kg) | 5.43 | 5.46 | 0.11 | ns |
| Weight gain from 6-24 h (kg) | 0.32 | 0.38 | 0.09 | ns |
| Weaning age (d) | 121.2 | 120.4 | 1.20 | Ns |
| Weaning weight (kg) | 35.9 | 34.6 | 0.73 | 0.032 |
| DLWG to weaning (kg/d) | 0.255 | 0.248 | 0.007 | Ns |

Behavioural data were collected on 209 lambs. There were no significant differences in neonatal vigour and time to first successful suckling between the treatments (Table 7). This may reflect the fact that feeding of DHA supplemented feed was only for a 4 week period prior to birth and longer feeding periods may be necessary.

TABLE 7
Lamb behaviour data

| | Control | +DHA | Sem | Sig |
|---|---|---|---|---|
| N | 97 | 112 | | |
| Time to first standing attempt (min) | 8.1 | 9.5 | 1.3 | ns |
| Time to first successful standing (min) | 15.1 | 15.4 | 1.6 | ns |
| Time to first successful suckling (min) | 31.9 | 30.9 | 3.3 | ns |
| Time from standing to suckling (min) | 16.8 | 14.4 | 3.3 | ns |

The DHA dose level used was adequate to give physiological change, as indicated by the increased gestation length. There was no evidence of adverse effect on colostrum yield (as with fish oil studies) since early lamb weight gain did not differ between treatments. Lamb mortality was very low and did not differ between treatments.

TABLE 1

| | Pregnancy supplement | | | | | | | |
|---|---|---|---|---|---|---|---|---|
| | DHA | DHA | CONTROL | CONTROL | | | | |
| | Lactation supplement | | | | | Statistical significance | | |
| | DHA | CONTROL | DHA | CONTROL | SEM | Gestation DHA | Lactation DHA | Interaction |
| Total litter size at birth | 11.0 | 13.8 | 13.2 | 11.9 | 0.66 | ns | ns | 0.005 |
| Born dead | 0 | 1.4 | 0.5 | 0.5 | 0.37 | ns | ns | ns |
| Born alive | 11.0 | 12.4 | 12.7 | 11.4 | 0.63 | ns | ns | .05 |
| Litter birth weight (kg) | 17.8 | 20.4 | 21.2 | 17.6 | 1.15 | ns | ns | .014 |
| Mean piglet birth weight (kg)‡ | 1.54 | 1.60 | 1.66 | 1.45 | 0.09 | ns | ns | ns |
| No after fostering | 11.0 | 12.0 | 11.7 | 11.5 | 0.64 | ns | ns | ns |
| No of deaths before weaning | 0.6 | 1.6 | 1.9 | 2.0 | 0.55 | Ns (0.15) | ns | ns |
| No of deaths before weaning† | 1.1 | 1.4 | 1.8 | 2.0 | 0.42 | Ns (0.120 | ns | ns |
| No weaned | 9.6 | 10.3 | 9.9 | 9.4 | 0.57 | ns | Ns | ns |
| Age at weaning (days) | 29.6 | 28.0 | 28.0 | 28.6 | 0.63 | ns | Ns | ns |
| Litter weaning weight | 83.8 | 74.4 | 78.2 | 72.2 | 4.77 | ns | Ns(0.11) | Ns |
| Mean piglet weaning weight (kg)¶ | 8.5 | 7.6 | 8.1 | 7.6 | 0.32 | ns | 0.03 | ns |
| Sow feed intake (kg/day) | 6.3 | 5.9 | 6.4 | 6.2 | 0.33 | ns | ns | ns |

‡total litter size at birth as covariate
†initial litter size as covariate
¶litter size and age at weaning as covariate

TABLE 2

| | Pregnancy supplement | | | | | | | |
|---|---|---|---|---|---|---|---|---|
| | DHA | DHA | CONTROL | CONTROL | | | | |
| | Lactation supplement | | | | | Statistical significance | | |
| | DHA | CONTROL | DHA | CONTROL | SEM | Gestation DHA | Lactation DHA | Interaction |
| Age at start (days) | 29.6 | 28.0 | 28.3 | 28.6 | 0.37 | ns | ns | 0.009 |
| Weight at start (kg) | 8.8 | 7.6 | 8.1 | 7.8 | 0.26 | ns | 0.006 | 0.07 |
| Weight after 7 days (kg) | 11.0 | 9.8 | 10.0 | 9.6 | 0.32 | 0.06 | 0.02 | ns |
| Weight after 14 days (kg) | 14.4 | 13.0 | 13.0 | 12.4 | 0.42 | 0.03 | 0.03 | Ns |
| Weight after 21 days (kg) | 18.2 | 17.2 | 17.0 | 16.2 | 0.53 | 0.05 | 0.09 | ns |
| Weight after 26 days (kg) | 21.8 | 20.6 | 20.4 | 19.2 | 0.59 | 0.03 | 0.05 | ns |
| Feed intake week 1 (g/pig/day) | 300 | 275 | 250 | 262 | 14.4 | 0.03 | ns | Ns |
| Feed conversion ratio week 1 | 0.94 | 0.87 | 0.97 | 1.04 | 0.05 | Ns (0.08) | ns | Ns |
| Feed intake in 26 days (g/pig/day) | 578 | 557 | 552 | 521 | 19.4 | Ns (0.12) | ns | ns |
| Feed conversion ratio in 26 days | 1.16 | 1.12 | 1.17 | 1.20 | 0.03 | Ns (0.08) | ns | ns |
| Liveweight gain week 1 (g/pig/day)‡ | 291 | 338 | 280 | 260 | 20.6 | 0.03 | ns | Ns (0.13) |
| Liveweight gain in 26 days (g/pig/day)‡ | 465 | 521 | 478 | 443 | 15.3 | 0.03 | ns | 0.007 |
| Feed intake week 1 (g/pig/day)‡ | 285 | 285 | 254 | 263 | 14.8 | 0.07 | ns | Ns |
| Feed conversion ratio week 1‡ | 0.99 | 0.84 | 0.96 | 1.03 | 0.05 | Ns (0.10) | ns | 0.05 |
| Feed intake in 26 days (g/pig/day)‡ | 545 | 579 | 557 | 527 | 18.9 | ns | ns | Ns (0.12) |
| Feed conversion ratio in 26 days‡ | 1.18 | 1.10 | 1.17 | 1.20 | 0.03 | ns | ns | Ns (0.07) |

‡weaning weight and age as covariates

TABLE 3
Crypthecodinium cohnii biomass

| Typical composition | g/100 g |
|---|---|
| Total fat | 46 |

Fatty acids (% of fat)

| | |
|---|---|
| 12:0 | 4.1 |
| 14:0 | 16.5 |
| 16.0 | 16.9 |
| 18:1n-9 | 10.2 |
| 22:6 (DHA) | 43.8 |
| Total Carbohydrate: | 18 |
| Total Protein: | 20 |

Amino acids (% of protein)

| | |
|---|---|
| methionine | 3.3 |
| cystine | 1.8 |
| lysine | 6.4 |
| phenylalanine | 3.3 |
| leucine | 6.5 |
| isoleucine | 4.1 |
| threonine | 5.4 |
| valine | 5.1 |
| histidine | 2.5 |
| arginine | 6.3 |
| glycine | 6.2 |
| aspartic acid | 13.2 |
| serine | 6 |
| glutamic acid | 12.9 |
| proline | 5.4 |
| hydrbxyproline | 0.1 |
| alanine | 7.7 |
| tryosine | 3.8 |
| Fibre: | 9.2 |
| Ash: | 2.8 |
| Residual moisture: | 4 |

1. An animal feed composition comprising a microbial biomass comprising a long chain omega-3 fatty acid.

2. The composition according to claim 1 wherein the animal feed is a gestation feed.

3. The composition according to claim 1 wherein the animal feed is a weaning feed or lactation feed.

4. The composition of claim 1, wherein the microbial biomass is an algal biomass.

5. The composition according to claim 4 wherein the algal biomass is derived from an edible algae.

6. The composition of claim 1, wherein the long chain omega-3 fatty acid is docosahexaenoic acid.

7. The composition of claim 1, wherein the long chain omega-3 fatty acid is present at a level of not less than 3 g/kg feed.

8. A method to improve the development of an animal species comprising:
i) administering to said animal a composition of claim 1, wherein said fatty acid is provided during late gestation and/or during lactation; and
ii) allowing an infant of said species to suckle on said animal administered said long chain fatty acid.

9. The method according to claim 8 wherein said animal is a livestock species.

10. The method according to claim 9 wherein said livestock species is a cow; sheep; goat; horse; mink; or pig.

11. The method according to claim 10 wherein said animal is a pig.

12. The method according to claim 11 wherein said pig is a pregnant pig.

13. The A method according to claim 10 wherein said animal is a sheep.

14. The method according to claim 13 wherein said sheep is pregnant.

15. The method according to claim 8 wherein said animal is a companion mammal.

16. The method according to claim 15 wherein said companion mammal is a dog; cat; hamster; mouse; rabbit; or pot bellied pig.

17. The method of claim 8, wherein said long chain fatty acid is a free fatty acid, or an ester thereof.

18. The method according to claim 17, wherein said free fatty acid is a triglyceride, diglyceride, monoglyceride, phospholipids, glycolipid, sulpholipid or sphingolipid.

19. The method of claim 8, wherein said long chain fatty acid is provided in late gestation.

20. The method of claim 8, wherein said long chain fatty acid is provided during lactation.

21. The method of claim 8, wherein said long chain fatty acid is provided up until weaning.

22. The method according to claim 19, wherein the fatty acid is docosahexaenoic acid, and wherein the docosahexaenoic acid is provided once daily during late gestation.

23. The method according to claim 20, wherein the fatty acid is docosahexaenoic acid, and wherein the docosahexaenoic acid is provided twice daily during lactation.

24.-27. (canceled)

28. The composition of claim 5, wherein the edible algae comprises Crypthecodinium spp; Phaedactylum spp; Isochrysis spp; Schizochytrium spp; Thaustochytrium spp; or Ulkenia spp.

29. The composition of claim 28, wherein the edible algae comprises Crypthecodinium spp.

30. The composition of claim 8, wherein the long chain fatty acid is derived from Crypthecodinium spp.
